(19) United States
(12) Patent Application Publication
(10) Pub. No.: US 2011/0045109 A1
(43) Pub. Date: Feb. 24, 2011

(54) NOVEL NUTRACEUTICAL COMPOSITIONS CONTAINING WHOLE-FRUIT ZANTHOXYLUM BUNGEANUM EXTRACT FOR COGNITION

(76) Inventors: Ann Fowler, Rheinfelden (CH); Regina Goralczyk, Grenzach-Wyhlen (DE); Claus Kilpert, Mannheim (DE); Annis Olivia Mayne-Mechan, Moehlin (CH); Hasan Mohajeri, Egg b. Zuerich (CH); Bernd Mussler, Lahr (DE); Adrian Wyss, Basel (CH)

Correspondence Address: NIXON & VANDERHYE, PC, 901 NORTH GLEBE ROAD, 11TH FLOOR, ARLINGTON, VA, 22203, US

(21) Appl. No.: 12/745,963

(22) PCT Filed: Dec. 3, 2008
(86) PCT No.: PCT/EP08/10226

§ 371 Date: Sep. 20, 2010

(30) Foreign Application Priority Data
Dec. 4, 2007 (EP) 07023420.8

(51) Int. Cl. A61K 36/758; A61P 25/00

(52) U.S. Cl. 424/769

(57) ABSTRACT

The invention relates to a novel nutraceutical composition containing Zanthoxlyum bungeanum whole-fruit extract as active ingredient. The term “nutraceutical” as used herein denotes usefulness in nutritional, pharmaceutical and veterinary fields of application. The compositions are useful for improvement of cognitive functions, such as learning, memory and alertness, psychotic stability and maintenance.

### FIELD OF THE INVENTION

The present invention relates to a novel nutraceutical composition or food additive comprising a whole-fruit (i.e. fruit and pericarp) extract of Zanthoxlyum bungeanum as active ingredient to improve cognitive functions such as learning, memory and alertness as well as relieving psychosocial pressure.

### BACKGROUND OF THE INVENTION

Memory, learning and alertness rely on neuronal circuits in the midbrain, especially in the hippocampus where information is processed and memory is consolidated. Mental performance and learning are dependent on synaptic plasticity; i.e. strengthening neuronal connections by the recruitment of new receptors, formation of new synapses and eventually the generation of new neuronal connections.

The formation of (long-term) memory and the efficient functioning of the brain depend on synthesis of new proteins for the reinforcement of communicative strength between neurones. The production of new proteins devoted to synapse reinforcement is triggered by chemical and electrical signals within neurones.

Long-term potentiation (LTP) is the term used to describe the long-lasting enhancement of synaptic transmission (hours in vitro, days or weeks in vivo) which occurs at particular synapses within the central nervous system (CNS) following a short, conditioning, burst of presynaptic electrical stimulation (approximately 100 Hz for 1 second). This phenomenon is widely considered to be one of the major mechanisms by which memories are formed and stored in the brain. LTP has been observed both in vitro and in living animals. Under experimental conditions, applying a series of short, high-frequency electrical stimuli to a synapse can potentiate the strength of the chemical synapse for minutes to hours. Most importantly, LTP contributes to synaptic plasticity in living animals, providing the foundation for a highly adaptable nervous system.

Two different receptor types are primarily involved in the process of LTP, namely the N-methyl-D-aspartate (NMDA) receptor complex and the α-amino-3-hydroxy-5-methyl-4-isoxazole propionic acid (AMPA) receptor. During LTP, the major excitatory neurotransmitter, glutamate, is released from the presynaptic neuron, binds to and activates the AMPA receptor on the postsynaptic membrane, leading to its depolarisation. At resting membrane potentials, the NMDA receptor channel is blocked by magnesium ions, but depolarisation of the postsynaptic membrane removes this block, enabling NMDA receptor activation and subsequent entry of calcium into the cell. This rise in intracellular calcium is believed to activate protein kinases, leading to gene transcription and the construction of reinforcing proteins (Niehoff (2005), The Language of Life: How Cells Communicate in Health and Disease, 210-223) and resulting in enhanced sensitivity of the AMPA receptor, thus further facilitating neurotransmission and maintenance of LTP.

NMDA receptors are composed of assemblies of NR1- and NR2-subunits; the glutamate binding domain is formed at the junction of these subunits. In addition to glutamate, the NMDA receptor requires a co-agonist, glycine, in order to modulate receptor function. The glycine binding site is found on the NR1 subunit, while the NR2 subunit possesses a binding site for polyamines, regulatory molecules that modulate the functioning of the NMDA receptor.

There is an increasing interest in the development of compounds, as well as nutraceutical compositions, that may be used to improve learning, memory and alertness, in both elderly and young people, individuals who need especially high memory and attention in their daily work, including students, construction workers, drivers, pilots, physicians, salespeople, executives, housewives, “high performance professionals” and people who are under mental or daily stress as well as persons who are prone to psychiatric instability, such as schizophrenia.

Thus, a compound or nutraceutical composition which enhances NMDA receptor function and enables improvements in learning, memory and alertness would be highly desirable. WO03039570 discloses the use of an extract of Pericarpium zanthoxyli (specifically the rind of dried fruits of Zanthoxlyum bungeanum) for protecting brain cells and improving memory. The extract is prepared by extraction with an organic solvent/water mixture, preferably by extracting with a low alcohol (1-4 carbon atoms), acetone, chloroform, methylene chloride, ether, ethyl acetate, or mixtures thereof. The extract of the present invention differs from that disclosed in WO03039570, in that the present invention utilizes a water extract prepared from the whole fruit, which is then re-extracted with alcohol/ether mixtures.

U.S. Pat. No. 6,419,950 (Bombardelli et al.) also describes the use of a pericarp extract of Zanthoxylum bungeanum, for various analgesic and cosmetic uses. The extract of the present invention differs from that disclosed in Bombardelli et al, in that the present invention utilizes a water extract prepared from the whole fruit, which is then re-extracted with alcohol/ether mixtures. Also, the uses of the present extract differ from the Bombardelli et al extract.

### DETAILED DESCRIPTION OF THE INVENTION

It has been found, in accordance with this invention, that an extract of the whole fruit (i.e. pericarp and fruit) of Zanthoxlyum bungeanum has the ability to induce LTP, which is important in memory formation and memory consolidation. As such, the Zanthoxlyum bungeanum whole-fruit extract is useful in enhancing cognitive functions.

Therefore one aspect of the invention is a novel nutraceutical composition, comprising Zanthoxlyum bungeanum whole-fruit extract to enhance cognitive functions.

The whole fruit extract of Zanthoxlyum bungeanum is also an aspect of this invention.

### BRIEF DESCRIPTION OF THE FIGURES

FIG. 1a is a graph showing the error rate in the stepdown test; a: significant differences to vehicle treated age-matched littermates during the washout period.

FIG. 1b is a graph showing the step-down behavioral testing, duration of latency to escape electric shock a: significant difference to vehicle treated age-matched littermates during the washout period b: significant difference to Ginkgo treated positive controls during the washout period.

FIG. 2: is a graph showing the latency period to find the hidden platform in the Morris water maze during training days.

### ZANTHOXLYUM BUNGEANUM EXTRACT

The pericarp of the fruit of a number of species in the genus Zanthoxlyum is commonly known as Sichuan (or Szechuan) pepper, which is widely used for flavouring cooking, particularly in Sichuan, China, in addition to Tibet, Bhutan, Nepal, Japan and other Asian countries. Those species most commonly used include: Zanthoxylum bungeanum Max. (China), Z. piperitum DC=Z. sansho (Central/Eastern China, Japan, Korea), Z. simulans Hance=Z. bungei (China, Taiwan) and Z. schinifolium Sieb. et Zucc. (China, Korea). Related species further include: Z. nitidum Roxb. (DC) (China, peninsular South East Asia), Z. rhetsa Pierre var. budranga Pier.=Z. limonella (Northwestern India, peninsular South East Asia), Z. armatum DC=Z. alatum Roxb. (Himalaya, peninsular South East Asia, East Asia), Z. avicennae (Lamk) DC=Z. tidorense (China, peninsular South East Asia, Indonesia) and Z. acanthopodium DC (eastern Himalaya, China, peninsular South East Asia, Sumatra, Indonesia) (see http://www.uni-graz.at/˜katzer/engl/Zantpip.html).

As used throughout the specification and claims, the term “Zanthoxlyum bungeanum whole-fruit extract” means that the extract comprises both the pericarp and the entire fruit.

The Zanthoxlyum bungeanum whole-fruit extract should be acceptable for use in a nutraceutical composition for animal or human consumption. Thus the solvent to be used during the re-extraction process should be approved by the various regulatory agencies for the intended use.

A typical Zanthoxlyum bungeanum whole-fruit extract prepared via solvent extraction methods will contain, for example, β-pinene, 1,8-terpinene, cis-piperitol acetate, oleic acid, palmitic acid, 4-terpineol, α-sanshool, γ-sanshool, hydroxy-α-sanshool, hydroxy-β-sanshool, hydroxy-γ-sanshool, α-pinene, sabinene, myrcene, β-phellandrene, linalool, isopulegol, terpinene-4-ol, α-terpineol, piperitone, α-hydroxy-4,6-dimethoxyacetophenone, kokusagine, skimmianine, haplopine, herniarin, n-nonacosane, o-cymene, xanthoxylin, hyperin, quercitrin, quercetin, isiquercitrin, rutin, myrecitin, myricitrin, kaempferol, luteolin.

In a preferred extract, the fruit and pericarp are crushed and subjected to a hot water extraction and subsequently dried either via evaporation or by spray-drying. The resulting extract is then re-extracted using an alcohol, such as methanol. This extract, made by this method is also an aspect of the present invention.

Conditions Improved by this Invention:

In the context of this invention “treatment” also encompasses co-treatment as well as prevention, lessening of symptoms, extending the time of onset, and ameliorating severity. Prevention also refers to maintaining a healthy state.

Throughout this specification and claims, the term “improved cognitive functions” is meant to refer to the conditions of supporting and maintaining cognitive wellness and balance, such as:
Enhanced learning, including:
   language processing
   problem solving
   intellectual functioning
Ability to cope with psychosocial burdens
Enhanced attention and concentration
Enhanced memory and the capacity for remembering, especially short-term memory
Enhanced mental alertness and mental vigilance, reduction of mental fatigue
Stabilisation of mental status including:
   Relieving post-partum conditions
   Relieving psychological burden due to separation of partners, children, death of beloved people or marital problems
   Relieving problems associated with change of domicile, work or similar events
   Relieving stressful conditions following a traffic accident or other negative social pressure
Stress relief, including:
   treatment, prevention and alleviation of symptoms related to work overload, exhaustion and/or burn out
   increased resistance or tolerance to stress
   favouring and facilitating relaxation in normal healthy individuals
“Condition improvement”, including:
   reducing irritability and tiredness
   reducing, preventing or alleviating physical and mental fatigue
   promoting good-quality sleep, that is to act against insomnia and sleep disorders and to increase energy in more general terms, in diseased or normal healthy individuals.

In a preferred aspect of the present invention the composition may be used as nutritional supplements, particularly for people who may feel a special need for enhanced cognitive function and/or psychosocial support. A non-exhaustive list of people who would benefit from enhanced cognitive function would include:
elderly people,
students or persons who are preparing for exams,
children who are engaged in a great deal of learning, i.e. infants, toddlers, pre-school children and school children,
construction workers, or those operating potentially dangerous machinery,
truck drivers, pilots, train drivers, or other transportation professionals,
air traffic controllers,
salespeople, executives, and other “high performance professionals”
police officers and military personnel,
housewives,
   or for anyone exposed to high amounts of stress in their daily work or who needs especially high attention/concentration/high mental and psychological performance in their daily work, such as those participating in sports, chess players, golfers, professional performers (actors, musicians and the like).

To achieve these improvements, administration over several days (for example at least six or ten days) is recommended, and administration daily for several weeks is generally preferred.

Aside from applications for humans, the compositions of this invention have additional uses in the veterinary world. Animals which can benefit from enhanced cognitive function include those animals which are subject to stressful conditions. Such conditions occur, for example, after capture or transport or may be due to housing conditions (such as change of domicile or owner), when the animals develop analogous disorders and are distressed or aggressive, or display stereotypic behaviour, or anxiety and obsessive-compulsive behaviour. Animals which are subject to stress would also include those which are racing animals (e.g. dogs, horses, camels), or used in various sports, performing animals (such as circus animals and those appearing on stage, television or in the movies) and horses which perform dressage and other highly disciplined routines.

Preferred “animals” are pets or companion animals and farm animals. Examples of pets are dogs, cats, birds, aquarium fish, guinea pigs, (jack) rabbits, hares and ferrets. Examples of farm animals are aquaculture fish, pigs, horses, ruminants (cattle, sheep and goats) and poultry.

### Nutraceutical Uses/Formulations/Dosages

The term “nutraceutical” as used herein denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Moreover, a multi-vitamin and mineral supplement may be added to nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions according to the present invention may be in any galenic form that is suitable for administering to the body, especially in any form that is conventional for oral administration, e.g. in solid forms such as (additives/supplements for) food or feed, food or feed premix, fortified food or feed, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or ligninsulfonate. Examples for other application forms are those for transdermal, parenteral or injectable administration. The dietary and pharmaceutical compositions may be in the form of controlled (delayed) release formulations.

Examples of food are dairy products including, for example, margarines, spreads, butter and cheese.

Examples of fortified food are cereal bars, bakery items, such as cakes and cookies, and potato chips or crisps.

Beverages encompass non-alcoholic and alcoholic drinks as well as liquid preparations to be added to drinking water and liquid food. Non-alcoholic drinks are e.g. soft drinks, sports drinks, fruit juices, lemonades, teas and milk-based drinks. Liquid foods are e.g. soups and dairy products. The nutraceutical composition containing Zanthoxlyum bungeanum whole-fruit extract may be added to a soft drink, an energy bar, or a candy, such that an adult consumes preferably 10 to 3000 mg, more preferably 50 to 2000 mg, and most preferably 100 to 1000 mg, of Zanthoxlyum bungeanum whole-fruit extract per serving.

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives. Preferred are formulations containing 2.5 to 1000 mg, more preferably 12.5 to 750 mg, and most preferably 25 to 500 mg of the Zanthoxlyum bungeanum whole-fruit extract derivate per administration unit, e.g. per tablet or capsule.

The following non-limiting Examples are presented to better illustrate the invention.

### Example 1

### Preparation of Zanthoxlyum bungeanum Whole-Fruit Extract

The original water extract was purchased from Shaanxi Jiahe Phytochem Co., Ltd. (Xi'an, China 710075). The water extract of whole fruits of Z. bungeanum was then dried via evaporation or spray-drying (10:1 ratio between raw material and extract) and re-extracted with alcohol/ether mixtures (e.g. methanol and methyl-tert-butyl ether (MTBE)) to achieve the specific extract used in the current invention.

### Example 2

### Hippocampal Slice Cultures

Seven-day-old Wistar rats were decapitated using a guillotine. In less than 1 minute the skull was opened, the cerebral hemispheres were separated and transferred and both hippocampi were dissected and transferred into ice cold buffer containing 137 mM NaCl, 5 mM KCl, 0.85 mM Na₂HPO₄, 1.5 mM CaCl₂, 0.66 mM KH₂PO₄, 0.28 mM MgSO₄, 1 mM MgCl₂, 2.7 mM NaHCO₃, 1 mM Kynurenic acid and 0.6% D-glucose.

Transverse hippocampal slices (400 μm) were prepared using a vibrating blade microtome (VT1200S; Leica Microsystems (Schweiz) AG, Heerbrugg, Switzerland) in the same buffer. Hippocampal slices were individually placed on a membrane insert (Millicell Culture Plate Inserts, 0.4 μm) and cultivated at 35° C., 5% CO₂, 95% humidity in a medium containing a 1:1 mixture of BME and MEM (both from Invitrogen) containing 25% heat-inactivated horse serum, 1× GlutaMAX, 1× Penicillin/Streptomycin, 0.6% glucose and 1 mM Kynurenic acid (Stoppini, Buchs and Muller (1991), J. Neurosci. Methods, 37(2), 173-82).

After 48 h in culture, synaptic NMDA receptors were activated by addition of Zanthoxlyum bungeanum whole-fruit extract for 15 min in 140 mM NaCl, 5 mM KCl, 1.3 mM CaCl₂, 25 mM HEPES (pH 7.3), 33 mM D-glucose and 0.02 mM bicuculline methiodide. Sarcosine (100 μM) and ALX5407 (20 nM) were used routinely as positive controls. An additional positive control comprised the addition of 200 μM glycine to sister cultures. After the treatments, sections were washed and fixed for immunohistochemistry. Markers of enhanced synaptic activity, normally associated with long-term potentiation, representing an ex vivo model of learning and memory were quantitated (see Table 1, below).

TABLE 1
Relative activation of synaptic markers after treatment with
Zanthoxlyum bungeanum whole-fruit extract in comparison
with sister cultures treated with buffer. The activation of any of
these markers (or a combination thereof) is observed in classical
LTP experiments. + shows a qualitative activation,
whereas − signifies a reduction in immunoreactivity. In the
GluR1 assay, data is shown as percentage of control values.

| Substance | pCREB | pMAPK | GluR1 |
|---|---|---|---|
| Zanthoxlyum bungeanum whole-fruit extract | + | − | 311% |

Treatment of hippocampal cultures with Zanthoxlyum bungeanum whole-fruit extract induced biochemical markers typical for LTP (pCREB: activated form of the cAMP response element binding protein; GluR1: cell surface presence of AMPA receptor 1). pMAPK (activated form of mitogen-activated protein kinase) was not activated by the Zanthoxylum bungeanum whole-fruit extract.

### Example 3

### Effects of Zanthoxylum Whole Fruit Extract in Two Traditional Rodent Models of Learning and Memory

### 1—Stepdown Test

Mice were subjected to an associative learning and memory paradigm, the stepdown test. The reaction box bottom was fitted with a 36V electric grid, and mice were individually placed in a reaction box. When animals receive an electric shock, their normal reaction is to jump up onto an insulated platform to avoid the pain stimulus. The majority of animals that jumped back onto the grid, would, upon receiving the electrical shock, rapidly jump back up on the platform. Animals were trained for 5 min, and the number of times each mouse was shocked, or made an error, was noted. This data constituted the learning data. Re-tests were done at 24 h (training day) and 48 h (test day), with these trials serving as the memory tests. The number of animals shocked in each group, the time prior to jumping down from the platform and the number of errors in the first 3 min were recorded. At five days after conclusion of training at day 8, memory decay was tested (washout test).

The study included 3 test groups (n=12 per group). All mice were administered test substances or vehicle via daily oral gavage (10 ml/kg) throughout the study. Treatment dose for Ginkgo biloba was 100 mg/kg BW, Zanthoxylum extract was tested at 3 doses 50 (low dose), 150 (mid dose), 450 (high dose) mg/kg BW). Rolipram was administered by interaperitoneal injection 30 min before testing (0.1 mg/kg body weight).

When compared to vehicle-treated littermates (negative control) Zanthoxylum treated animals exhibited a significant better learning and memory performance during the test and memory phase and after the wash-out period.

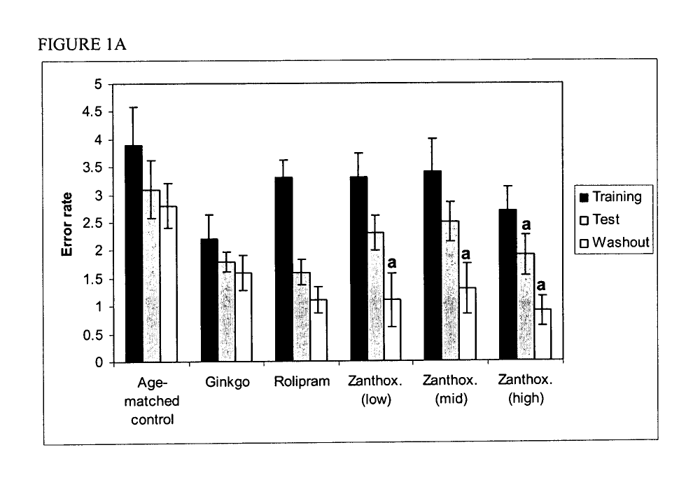

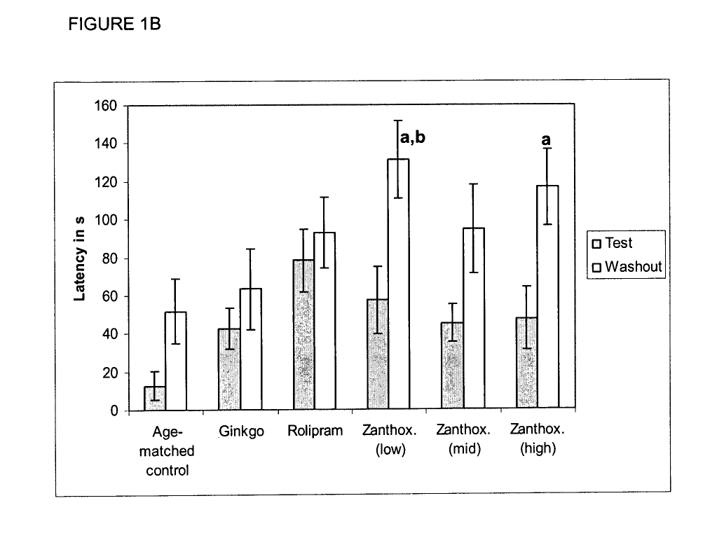

FIG. 1a shows the error rate in the stepdown test; a: significant differences to vehicle treated age-matched littermates during the washout period. These results show that all animals learned the task equally well, but the Zanthoxylum treated mice retained the memory significantly longer than the control animals.

FIG. 1b shows the duration of latency to escape electric shock during the step-down behavioral testing, a: significant difference to vehicle treated age-matched littermates during the washout period b: significant difference to Ginkgo treated positive controls during the washout period. Again, Zanthoxylum treated mice retained the memory significantly longer than other groups.

In conclusion, mice treated with Zanthoxylum whole fruit extract exhibited significantly better memory performance than their age-matched controls. Zanthoxylum treated mice performed not only better than the age-matched controls but also better than the Ginkgo treated positive controls.

### Example 4

Effects of zanthoxylum Whole Fruit Extract in Two Traditional Rodent Models of Learning and Memory

### 2—Morris Water Maze

The Morris water maze is one of the best accepted paradigms to test spatial memory in rodents. Mice have to swim in a round pool and have to search a hidden platform. As orientation help they use visual cues in the experimentation room.

In this experiment we used a Morris water maze, diameter=1.5m, with a hidden platform (10 cm×10 cm) which was placed 10 cm from the border at the 10 o'clock direction. Mice were adapted and trained twice to swim to the platform at day 0.

From day 1-4, mice were trained twice per day, and the time to locate the platform was recorded. The average time of each day's two tests was calculated. In addition the numbers of platform crossings were recorded as well. The first 3 days scores were taken as Training Scores. The fourth day's two test was taken as Testing Scores.

During the training sessions only the groups which received the low and medium zanthoxylum concentration performed significantly better. On day 4 when the probe trial took place, the group with the middle Zanthoxylum concentration showed a significant shorter latency time compared to the age-matched control animals suggesting that treatment with Zanthoxylum extract affects both learning and memory performance of the animals.

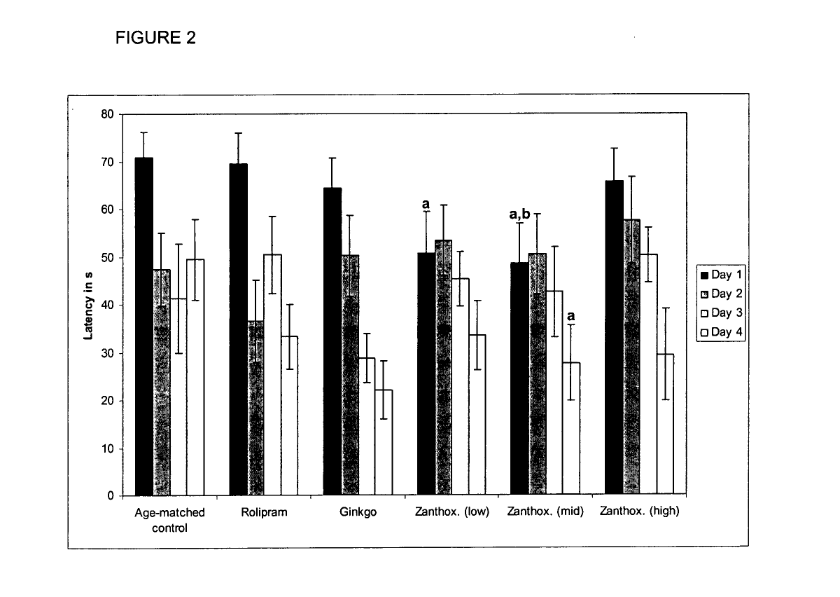

Results are shown in FIG. 2, demonstrating the Latency period to find the hidden platform in the Morris water maze during training days. a: significant difference to vehicle treated age-matched littermates during the first day emphasizing that treatment with low dose Zanthoxylum extract improved significantly the learning performance of the animals. At the medium dose, Zanthoxylum treatment even improved the learning behavior more that that observed for Ginkgo treated animals. On day 4, during the probe trial, a significant difference was observed between the Zanthoxylum mid-dose group and vehicle treated age-matched littermates again showing that Zanthoxylum extract improved significantly the memory performance of the animals.

### Example 5

### Preparation of a Soft Gelatine Capsule

A soft gelatine capsule (200 mg) may be prepared comprising the following ingredients:

| Ingredient | Amount per Capsule |
|---|---|
| Zanthoxlyum bungeanum whole-fruit extract | 200 mg |
| Lecithin | 50 mg |
| Soy bean oil | 250 mg |

Two capsules per day for 3 months may be administered to a human adult. Cognitive functions, alertness, memory and the ability to focus on work are seen to improve.

### Example 6

### Preparation of a Fortified Non-Baked Cereal Bar

| Ingredient | Amount [g] |
|---|---|
| Zanthoxlyum bungeanum whole-fruit extract | 0.95 |
| Sugar | 114.55 |
| Water | 54.0 |
| Salt | 1.5 |
| Glucose syrup | 130.0 |
| Invert sugar syrup | 95.0 |
| Sorbitol Syrup | 35.0 |
| Palm kernel fat | 60.0 |
| Baking fat | 40.0 |
| Lecithin | 1.5 |
| Hardened palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skimmed milk powder | 45.0 |
| Apple flavour 74863-33 | 2.0 |
| Citric acid | 5.0 |
| Total amount | 1000 |

The Zanthoxlyum bungeanum whole-fruit extract is premixed with skimmed milk powder and placed in a planetary bowl mixer. Cornflakes and rice crispies are added and the total is mixed gently. Then the dried and cut apples are added. In one cooking pot, sugar, water and salt are mixed in the amounts given above (solution 1). In a second cooking pot, glucose, invert sugar- and sorbitol-syrups are mixed in the amounts given above (solution 2). The fat phase constitutes a mixture of baking fat, palm kernel fat, lecithin and emulsifier. Solution 1 is heated to 110° C. Solution 2 is heated to 113° C. and then cooled in a cold water bath. Subsequently, solutions 1 and 2 are combined. The fat phase is melted at 75° C. in a water bath, then added to the combined mixture of solutions 1 and 2. Apple flavour and citric acid are added to the liquid sugar/fat mix. The liquid mass is added to the dry ingredients and mixed well in the planetary bowl mixer. The mass is put on a marble plate and rolled to the desired thickness. The mass is cooled down to room temperature and cut into pieces. The non-baked cereal bar contains ca. 29 mg Zanthoxlyum bungeanum whole-fruit extract per serving (30 g). For general well-being and energising 1-2 cereal bars may be eaten per day.

### Example 7

### Preparation of Zanthoxlyum bungeanum Whole-Fruit Extract-Coated Potato Crisps

Potatoes are sliced very thinly, drizzled with olive oil and baked in an oven (425° F.) until brown and crispy (about 30 minutes). Alternatively, the thin slices are deep fried in vegetable oil (350-375° F.) until crispy. Subsequently, the crisps are coated with a composition comprising sea salt and Zanthoxlyum bungeanum whole-fruit extract.

### Example 8

Dry Dog Feed containing Zanthoxlyum bungeanum Whole-Fruit Extract

A commercial basal diet for dogs (e.g. Mera Dog “Brocken”, MERA-Tiernahrung GmbH, Marienstraβe 80-84, D-47625 Kevelaer-Wetten, Germany) is sprayed with a suspension of corn oil containing Zanthoxlyum bungeanum whole-fruit extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a dog a daily dose of 5 mg Zanthoxlyum bungeanum whole-fruit extract per kg body weight. The food composition is dried to contain dry matter of about 90% by weight. For an average dog of 10 kg body weight to consume approx. 200 g dry feed per day, the dog food contains approx. 250 mg Zanthoxlyum bungeanum whole-fruit extract per kg food. For heavier dogs, the feed mix is prepared accordingly. For reduction of stress, fear and aggressiveness in dogs, the food can be given to dogs in animal shelter farms on a regular basis. Before veterinarian visits or stays in veterinarian clinics or holiday separation, the food is given at least one week before, during the stressful event and one week thereafter.

### Example 9

Wet Cat Food Containing Zanthoxlyum bungeanum Whole-Fruit Extract

A commercial basal diet for cats (e.g. Happy Cat “Adult”, Tierfeinnahrung, Südliche Hauptstraβe 38, D-86517 Wehringen, Germany) is mixed with a suspension of corn oil containing Zanthoxlyum bungeanum whole-fruit extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a cat a daily dose of 10 mg Zanthoxlyum bungeanum whole-fruit extract per kg body weight. For an average cat of 5 kg of body weight to consume approx. 400 g of wet food, the cat food contains 125 mg Zanthoxlyum bungeanum whole-fruit extract per kg food. The food composition is dried to contain dry matter of about 90% by weight. For reduction of stress, fear and aggressiveness in cats, the food can be given to cats in animal shelter farms on a regular basis. Before veterinarian visits or stays in veterinarian clinics, the food is given at least one week before, during the stressful event and one week thereafter.

### Example 10

### Dog Treats Containing Zanthoxlyum bungeanum Whole-Fruit Extract

Commercial dog treats (e.g. Mera Dog “Biscuit” for dogs as supplied by Mera Tiernahrung GmbH, Marienstral3e 80-84, 47625 Kevelaer-Wetten, Germany) are sprayed with a suspension of corn oil containing Zanthoxlyum bungeanum whole-fruit extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to the treats 0.5-5 mg Zanthoxlyum bungeanum whole-fruit extract per g treats. The food composition is dried to contain dry matter of about 90% by weight. To reduce fear and tension, the treat can be given during the day in addition to the food, or when feeding is not warranted, i.e. upon travels, for up to 5 times per day.

### Example 11

### Cat Treats Containing Zanthoxlyum bungeanum Whole-Fruit Extract

Commercial cat treats (e.g. Whiskas Dentabits for cats as supplied by Whiskas, Masterfoods GmbH, Eitzer Str. 215, 27283 Verden/Aller, Germany) are sprayed with a suspension of corn oil containing Zanthoxlyum bungeanum whole-fruit extract, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-35 from DSM Nutritional Products Ltd, Kaiseraugst, Switzerland) in an amount sufficient to administer to the treats 0.5-5 mg Zanthoxlyum bungeanum whole-fruit extract per g treats. The food composition is dried to contain dry matter of about 90% by weight. To reduce fear and tension, the treat can be given during the day in addition to the food, or when feeding is not warranted, i.e. upon travels, for up to 5 times per day.

1. A method of improving cognitive function and/or psychological well-being in an animal or human comprising administering a cognitive function-improving amount of Zanthoxlyum bungeanum whole-fruit extract.

2. A method according to claim 1 comprising administering Zanthoxlyum bungeanum whole-fruit extract in an amount effective in inducing long-term potentiation.

3. A method according to claim 1 wherein the cognitive function and/or psychological well-being is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilisation of mental status, a stress reliever, work-overload stress, stress-related exhaustion and/or burn out, and to promote relaxation.

4. A composition used in the manufacture of a nutraceutical or medicament for improving cognitive function in an animal or human, which comprises a cognitive function-improving amount of Zanthoxlyum bungeanum whole-fruit extract.

5. A composition according to claim 4, wherein the composition comprises Zanthoxlyum bungeanum whole-fruit extract in an amount effective in inducing long-term potentiation.

6. A composition according to claim 4 wherein the cognitive function is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilisation of mental status, a stress reliever, work-overload stress, stress-related exhaustion and/or burn out, and to promote relaxation.

7. A nutraceutical comprising a whole-fruit extract of Zanthoxylum bungeanum.

8. A whole-fruit extract of Zanthoxylum bungeanum prepared by the process of:
exposing the fruit and pericarp to water to obtain an aqueous extraction;
removing the water to obtain an aqueous extraction residue; and
re-extracting the aqueous extraction residue with organic solvents.

9. An extract according to claim 8 wherein the organic solvents are methanol and methyl-tert-butyl ether.
